(19) United States
(12) Patent Application Publication
(10) Pub. No.: US 2023/0098734 A1
(43) Pub. Date: Mar. 30, 2023

(54) ELECTRONIC DEVICE AND CONTROL METHOD THEREOF

(71) Applicant: SAMSUNG ELECTRONICS CO., LTD., Suwon-si (KR)

(72) Inventors: Illia FEDORIN, Kiev (UA); Kostyantyn SLYUSARENKO, Kiev (UA)

(21) Appl. No.: 17/886,979

(22) Filed: Aug. 12, 2022

(30) Foreign Application Priority Data
Sep. 30, 2021 (KR) 10-2021-0129739

(51) Int. Cl. A61B 5/00

(52) U.S. Cl. CPC A61B 5/7278; A61B 5/7246; A61B 5/7264; A61B 5/742; A61B 5/4806

(57) ABSTRACT

Disclosed herein is an electronic device and a control method thereof. The control method of an electronic device includes: obtaining a bio-signal from at least one sensor, determining a first physiological parameter based on the bio-signal, estimating a second physiological parameter including a specified correlation with the first physiological parameter, and providing information about the estimated second physiological parameter.

### CROSS-REFERENCE TO RELATED APPLICATION

This application is based on and claims priority under 35 U.S.C. § 119 to Korean Patent Application No. 10-2021-0129739, filed on Sep. 30, 2021, in the Korean Intellectual Property Office, the disclosure of which is incorporated by reference herein in its entirety.

### BACKGROUND

### 1. Field

The disclosure relates to an electronic device configured to estimate a physiological parameter of a user, and a control method thereof.

### 2. Description of Related Art

Electronic devices, such as smart watches, configured to measure a bio-signal of a user are introduced into the market. In other words, the electronic device may include various sensors configured to measure a bio-signal of a user. For example, the electronic device may measure a bio-signal such as heart rate, pulse rate, blood oxygen saturation, blood pressure, or blood sugar. The electronic device may analyze the bio-signal of the user to determine a physiological condition of a user. Analysis of the physiological condition of a person requires a number of independent and interdependent physiological parameters such as heart rate, heart rate variability, blood pressure, respiration rate and body temperature.

In order to directly measure these physiological parameters, various sensors that are complex, expensive and energy-consuming are required. Further, when many types of sensors are included in the electronic device, a price of the electronic device may increase, and difficulties may arise in the production of the electronic device. In addition, because various sensors operate together, a battery efficiency of the electronic device may decrease and memory resources may be wasted.

### SUMMARY

Embodiments of the disclosure provide an electronic device capable of estimating an interdependent physiological parameter correlated with a physiological parameter directly obtainable from a sensor, and a control method thereof.

In accordance with an example embodiment of the disclosure, a method of controlling an electronic device includes: obtaining a bio-signal from at least one sensor, determining a first physiological parameter based on the bio-signal, estimating a second physiological parameter including a specified correlation with the first physiological parameter, and providing information about the estimated second physiological parameter.

The second physiological parameter may include at least one of biometric data or a physiological condition correlated with the first physiological parameter and not obtained by the sensor.

The estimation of the second physiological parameter may include estimating the second physiological parameter dependent on the first physiological parameter using an artificial intelligence model.

The estimation of the second physiological parameter may include estimating a plurality of different second physiological parameters from the first physiological parameter using the artificial intelligence model.

The artificial intelligence model may include a least one of a deep neural network (DNN) model, a convolutional neural network (CNN) model, a recurrent neural network (RNN) model, or a long short-term memory (LSTM) model.

The providing of the information about the second physiological parameter may include determining whether an additional sensor is needed for directly measuring the second physiological parameter, by comparing the estimated second physiological parameter with a specified reference value, and providing information about the additional sensor.

The obtaining of the bio-signal and the estimation of the second physiological parameter may be continuously performed at a specified interval.

The information about the second physiological parameter may include personalized feedback information based on an analysis of the second physiological parameter that changes over time.

The personalized feedback information may include at least one of potential risk information about the physiological condition or recommended activity information about the physiological condition.

The method may further include pre-processing the bio-signal, and the pre-processing may include data filtering, noise removal, motion artifact removal, and normalization and standardization of personalized data for variability reduction.

In accordance with an example embodiment of the disclosure, an electronic device includes: a display, at least one sensor configured to obtain a bio-signal, and a processor electrically connected to the display and the at least one sensor. The processor is configured to: determine a first physiological parameter based on the bio-signal, estimate a second physiological parameter including a specified correlation with the first physiological parameter, and control the display to provide information about the estimated second physiological parameter.

The processor may be configured to estimate the second physiological parameters dependent on the first physiological parameter using an artificial intelligence model.

The processor may be configured to estimate a plurality of different second physiological parameters from the first physiological parameter using the artificial intelligence model.

The processor may be configured to determine whether an additional sensor is needed for directly measuring the second physiological parameter, by comparing the estimated second physiological parameter with a specified reference value, and the processor may be configured to control the display to provide information about the additional sensor.

The processor may be configured to control the sensor to obtain the bio-signal and configured to estimate the second physiological parameter at a specified interval in a continuous manner.

The processor may be configured to pre-process the bio-signal by perforr ping data filtering, noise removal, motion artifact removal, and normalization and standardization of personalized data for variability reduction.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example configuration of an electronic device according to various embodiments;

FIG. 2 is a flowchart illustrating an example method of controlling the electronic device according to various embodiments;

FIG. 3 is a diagram illustrating an example of physiological parameters directly obtainable from bio-signals of a biosensor included in the electronic device according to various embodiments;

FIG. 4 is a diagram illustrating an example of physiological parameters estimated from the directly obtained physiological parameters of FIG. 3 according to various embodiments;

FIG. 5 is a diagram illustrating an example of a neural network architecture used to estimate the physiological parameters according to various embodiments;

FIG. 6 is a graph illustrating a correlation between a respiratory event during sleep and a blood oxygen saturation according to various embodiments;

FIG. 7 is a graph illustrating a blood oxygen saturation estimated from a respiratory event during sleep by a method according to various embodiments;

FIG. 8 is a graph illustrating the blood oxygen saturation estimated from the respiratory event during sleep by the method according to various embodiments;

FIG. 9 is a diagram illustrating an example of providing the estimated physiological parameter and information thereon by the electronic device according to various embodiments; and

FIG. 10 is a diagram illustrating an example of providing the estimated physiological parameter and information thereon by the electronic device according to various embodiments.

### DETAILED DESCRIPTION

Various example embodiments of the disclosure may be described with reference to accompanying drawings. The various example embodiments and the terms used therein are not intended to limit the technology disclosed herein to specific forms, and the disclosure should be understood to include various modifications, equivalents, and/or alternatives to the corresponding embodiments.

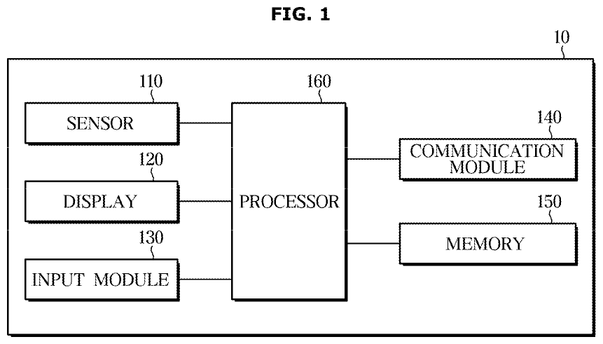

FIG. 1 is a block diagram illustrating an example configuration of an electronic device according to various embodiments.

Referring to FIG. 1, an electronic device 10 according to an embodiment may include a sensor 110, a display 120, an input module (e.g., including input circuitry) 130, a communication module (e.g., including communication circuitry) 140, a memory 150, and a processor (e.g., including processing circuitry) 160. The processor 160 may be electrically connected to components of the electronic device 10.

The electronic device 10 may be worn on or in contact with the user's body so as to measure a bio-signal of a user, and to obtain user's physiological information.

The electronic device 10 may include any device including the sensor 110 configured to measure a bio-signal of a user. For example, the electronic device 10 may include a wearable device such as a watch, a ring, a bracelet, an anklet, a necklace, glasses, a contact lens, a head-mounted-device (HMD), or the like. The electronic device 10 may include a computing device such as a laptop computer, a desktop computer, and a tablet, and may include a mobile device such as a smart phone. However, the electronic device 10 is not limited to the above-described devices.

The sensor 110 may be configured to non-invasively obtain various types of bio-signals. The sensor 110 may be implemented as a plurality of modules or as an integrated module. For example, the sensor 110 may include at least one of a photoplethysmogram (PPG) sensor 112, an electrocardiogram (ECG) sensor, a galvanic skin response (GSR) sensor, an electroencephalogram (EEG) sensor, and a pulse oximeter (PO) sensor 113, a bioelectrical impedance analysis (BIA) sensor, a body temperature sensor, a gesture sensor, a gyroscope, an acceleration sensor 111 and/or an audio sensor 115. The audio sensor 115 may correspond to a microphone. In addition, the sensor 110 may include sensors configured to obtain a bio-signal of a user. That is, the sensor 110 may perform various sensing functions.

The sensor 110 may transmit the obtained bio-signal to the processor 160. The processor 160 may control the sensor 110 to obtain a bio-signal at a predetermined interval or to obtain a bio-signal based on a user's input. In addition, the processor 160 may activate some or all of the plurality of sensors 110, if necessary. For example, the processor 160 may basically activate the acceleration sensor 111 and the PPG sensor 112, and if necessary, the processor 160 may additionally activate the PO sensor 113, the ECG sensor 114 and/or the audio sensor 115.

The processor 160 may include various processing circuitry and pre-process the bio-signal obtained by the sensor 110. An effective bio-signal may be obtained through the pre-processing of the bio-signal. For example, the processor 160 may perform data filtering, noise removal, motion artifact removal on the obtained bio-signal, and normalization and standardization of personalized data for variability reduction. The pre-processing of the bio-signal may be performed in a time domain and a frequency domain, and specific characteristics of the bio-signal may be obtained through the pre-processing of the bio-signal.

The processor 160 may determine a first physiological parameter using the preprocessed bio-signal. That is, the first physiological parameter may refer, for example, to a physiological parameter that may be directly derived from the bio-signal of the sensor 110. For example, the first physiological parameter may include at least one of heart rate, heart rate variability, respiration rate, blood oxygen saturation, electrocardiogram, and photoplethysmography, ballistocardiography, body temperature, and/or activity information. The activity information may be determined from a signal sensed by a motion sensor such as the acceleration sensor 111 or the gyroscope. An algorithm, a program, and/or software for determining physiological parameters corresponding to each of the plurality of sensors 110 may be stored in the memory 150.

Further, the processor 160 may estimate a second physiological parameter including a predetermined correlation with the first physiological parameter. For example, the second physiological parameter may include at least one of respiratory cycle, blood oxygen saturation, sleep apnea, hypopnea, various types of respiratory disorders, snoring, acute respiratory distress syndrome, and/or blood pressure. In addition, the second physiological parameter may include various biometric data and/or physiological conditions.

The second physiological parameter may include at least one of biometric data or a physiological condition that is correlated with the first physiological parameter and is not obtained by the sensor 110. The processor 160 may estimate the second physiological parameter dependent on the first physiological parameter using an artificial intelligence model. In other words, the processor 160 may estimate a plurality of different second physiological parameters from the first physiological parameter using the artificial intelligence model. For example, based on the user's movement detected by the acceleration sensor 111 and the user's heart rate detected by the PPG sensor 112, the processor 160 may estimate at least one of sleep apnea/hypopnea, snoring, heart rate disease, or blood oxygen saturation correlated with the user's movement and the user's heart rate.

Based on the plurality of first physiological parameters determined from the bio-signals obtained by the sensor 110, the processor 160 may estimate the second physiological parameter by applying a predetermined weight to the plurality of first physiological parameters. The weight may be determined according to the second physiological parameter to be estimated. That is, the weights to be applied to the plurality of first physiological parameters may be changed based on the second physiological parameter to be estimated.

The display 120 may provide visual information such as text, images, and graphic objects. For example, the display 120 may output at least one piece of information about the bio-signal of the user, the first physiological parameter, and/or the second physiological parameter. The information about the second physiological parameter may include personalized feedback information based on an analysis of the second physiological parameter that changes over time. For example, the personalized feedback information may include at least one piece of potential risk information about the physiological condition of the user or recommended activity information about the physiological condition of the user.

Further, the information about the second physiological parameter may include information about an additional sensor. The processor 160 may determine whether an additional sensor is needed for directly measuring the second physiological parameter, by comparing the estimated second physiological parameter with a predetermined reference value. The processor 160 may control the display 120 to provide information on the additional sensor. For example, in response to the estimated blood oxygen saturation (SpO2) being lower than a reference value (e.g., 95%), a message indicating that the direct measurement by the PO sensor 113 is required may be provided on the display 120.

The display 120 may be implemented as a liquid crystal display (LCD), an organic light emitting display (OLED), a quantum dot LED, a mini-LED, a micro-LED, or the like. In addition, the display 120 may include a touch sensor configured to detect a touch or a pressure sensor configured to measure an intensity of a force generated by the touch.

The input module 130 may include various input circuitry and receive a command or data from the outside (e.g., a user). For example, the input module 130 may include at least one of a switch, a mouse, a keyboard, a button, and a digital pen. In addition, the input module 130 may be implemented as a touch panel or a touch screen panel, and may be provided integrally with the display 120.

The communication module 140 may include various communication circuitry and establish a communication channel with an external device, and may support transmission and reception of data through the established communication channel. The communication module 140 may be implemented with various communication technologies supporting wired communication or wireless communication. For example, the communication technology such as Bluetooth, Wi-Fi, Radio Frequency (RF) communication, infrared communication, Ultra-Wide Band (UWB) communication, Near Field Communication (NFC), Zigbee, cellular communication, or a wide area network (WAN) may be applied to the communication module 140. In addition, the communication module 140 may further include a Global Positioning System (GPS) receiver configured to obtain location information.

The memory 150 may store various data used by at least one component (e.g., the processor 160) of the electronic device 10. Data may include software, programs, input data, and output data. The memory 150 may include at least one of a volatile memory and a non-volatile memory. The program may be stored as software in the memory 150 and may include an operating system, middleware, or an application.

The processor 160 may execute software or a program to control at least one other component (e.g., a hardware or software component) of the electronic device 10 connected to the processor 160, and the processor 160 may perform various data processing or calculations. As at least a part of data processing or calculation, the processor 160 may store commands or data received from other components (e.g., the sensor 110 or the communication module 140) in the memory 150, process the command or data stored in the memory 150, and store the result data in the memory 150. The processor 160 may include a central processing unit or an application processor.

The processor 160 may include a hardware structure specialized in processing of an artificial intelligence model. Artificial intelligence models may be generated through machine learning. The learning may be performed in the electronic device 10 itself in which the artificial intelligence model is performed, or may be performed through a separate server. A learning algorithm may include supervised learning, unsupervised learning, semi-supervised learning, or reinforcement learning, but is not limited thereto.

The artificial intelligence model may include a deep neural network (DNN) model, a convolutional neural network (CNN) model, a recurrent neural network (RNN) model, a long short-term memory (LSTM) model, a restricted Boltzmann machine (RBM), a deep belief network (DBN), a bidirectional recurrent deep neural network (BRDNN), a deep Q-network, or a combination of two or more of these, but is not limited thereto. The artificial intelligence model may additionally or alternatively include a software structure, in addition to the hardware structure.

In various embodiments, at least one of the above-described components may be omitted or one or more other components may be added to the electronic device 10. In various embodiments, some of these components may be integrated into one component. For example, the electronic device 10 may further include a power management module, a battery configured to supply power to at least one component of the electronic device 10, a sound output device such as a speaker, and a camera.

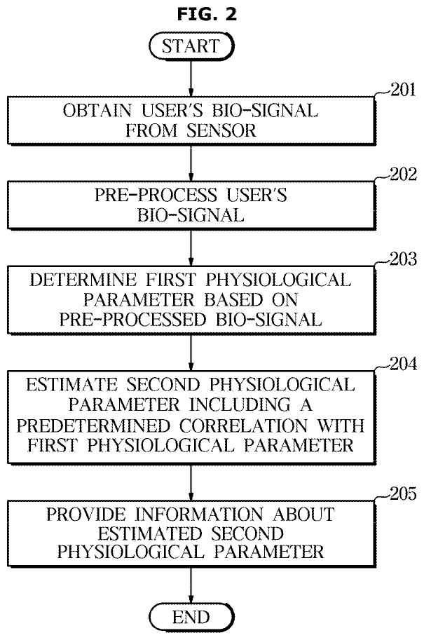

FIG. 2 is a flowchart illustrating an example method of controlling the electronic device according to various embodiments.

Referring to FIG. 2, the processor 160 of the electronic device 10 may obtain at least one bio-signal of a user from the sensor 110 (201). The sensor 110 may obtain various bio-signals by including various types of sensors, and may transmit the obtained bio-signals to the processor 160. For example, the sensor 110 may include the acceleration sensor 111 and the PPG sensor 112, and obtain an electrical signal corresponding to a user's movement and an electrical signal corresponding to a change in a blood volume in micro-vessels of a tissue, and transmit the electrical signals to the processor 160.

The processor 160 may pre-process the bio-signal obtained by the sensor 110 (202). An effective bio-signal may be obtained through the pre-processing of the bio-signal. For example, on the bio-signal of the acceleration sensor 111 and the bio-signal of the PPG sensor 112, the processor 160 may perform data filtering, noise removal, motion artifact removal and normalization and standardization of personalized data for variability reduction. Therefore, the processor 160 may extract the effective bio-signal.

The processor 160 may determine at least one of the physiological parameters using the pre-processed bio-signal (203). The first physiological parameter may refer, for example, to a physiological parameter that may be directly derived from the bio-signal of the sensor 110. For example, based on the pre-processed bio signal of the acceleration sensor 111 and the PPG sensor 112, the processor 160 may determine at least one of heart rate, heart rate variability, or respiration rate, and each of these may be determined as the first physiological parameter.

The processor 160 may estimate the second physiological parameter including a predetermined (e.g., specified) correlation with the first physiological parameter (204). The second physiological parameter may include at least one of biometric data or a physiological condition that is correlated with the first physiological parameter and is not obtained by the sensor 110. The processor 160 may estimate at least one of the second physiological parameters dependent on the first physiological parameter using the artificial intelligence model. For example, because there is a strong correlation between the bio-signals of the acceleration sensor 111 and the PPG sensor 112 and the blood oxygen saturation, the processor 160 may estimate a blood oxygen saturation using the artificial intelligence model based on the bio-signals of the acceleration sensor 111 and the PPG sensor 112. Further, the processor 160 may estimate sleep apnea/hypopnea, snoring, and heart rate disease based on the bio-signals of the acceleration sensor 111 and the PPG sensor 112.

Based on the plurality of first physiological parameters being determined from the bio-signals obtained by the sensor 110, the processor 160 may estimate the second physiological parameter by applying a predetermined weight to the plurality of first physiological parameters. The weight may be determined according to the second physiological parameter to be estimated. That is, the weights to be applied to the plurality of first physiological parameters may be changed based on the second physiological parameter to be estimated.

The processor 160 may control the display 120 to provide information about the estimated second physiological parameter (205). The information about the bio-signal measured by the sensor 110 and the first physiological parameter may also be provided. For example, at least one piece of potential risk information about the physiological condition of the user, recommended activity information about the physiological condition of the user, and information about an additional sensor for directly measuring the second physiological parameter may be provided.

Based on a sound output device such as a speaker included in the electronic device 10, information about the bio-signal, the first physiological parameter, and/or the second physiological parameter may also be provided through the sound output device.

As mentioned above, by estimating various physiological parameters from some bio-signals that are directly measured, the electronic device 10 may provide various physiological information to the user even without many biosensors.

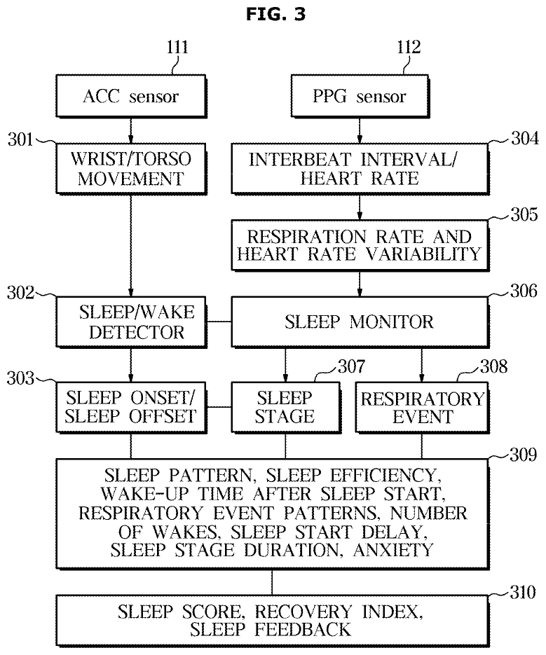

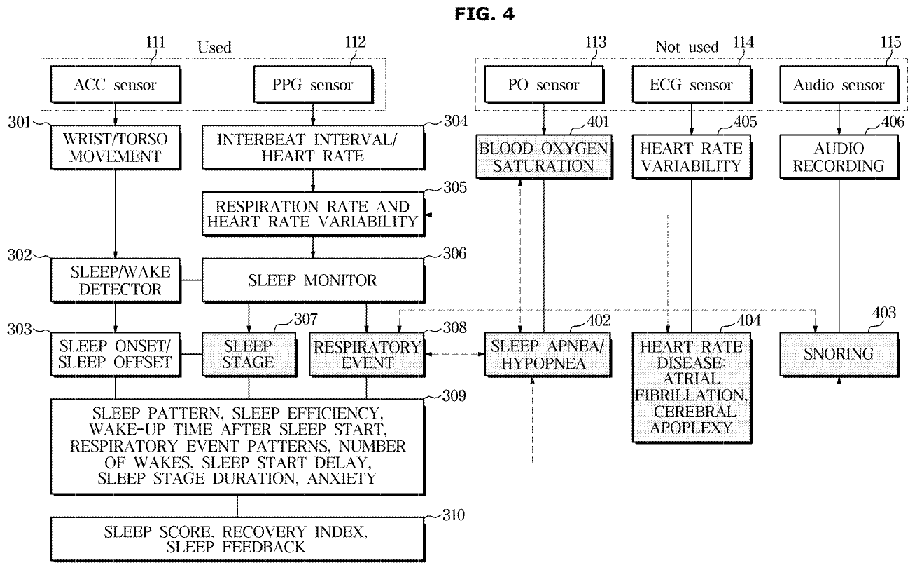

FIG. 3 is a diagram illustrating an example of physiological parameters directly obtainable from bio-signals of a biosensor included in the electronic device according to various embodiments. FIG. 4 is a diagram illustrating an example of physiological parameters estimated from the directly obtained physiological parameters of FIG. 3 according to various embodiments.

Referring to FIG. 3, the electronic device 10 may include the acceleration sensor 111 and the PPG sensor 112. The acceleration sensor 111 may detect a user's wrist/torso movement 301 and may output an electrical signal corresponding to the wrist/torso movement 301. The PPG sensor 112 may measure an interbeat interval and the heart rate 304, and may output an electrical signal corresponding to the interbeat interval and the heart rate.

The memory 150 may store algorithms or programs to process each of signal of the acceleration sensor 111 and the PPG sensor 112. The processor 160 may determine physiological parameters using the algorithms or programs. For example, the processor 160 may determine a sleep onset/sleep offset 303 from the wrist/torso movement 301 using a sleep/wake detector 302. In addition, the processor 160 may determine a respiration rate and heart rate variability 305 from the interbeat interval and heart rate 304 and determine a sleep stage 307 and a respiratory event 308 using a sleep monitor 306. The respiratory event may include apnea and hypopnea during sleep.

In addition, based on the sleep onset/sleep offset 303, the sleep stage 307 and/or the respiratory event 308, the processor 160 may determine physiological parameters 309 such as sleep pattern, sleep efficiency, wake-up time after sleep start, number of wakes, sleep start delay, respiratory event pattern, sleep stage duration and/or anxiety. Information on the physiological parameters 309 may be provided as sleep score, recovery index and/or sleep feedback shown in 310.

Referring to FIG. 4, a blood oxygen saturation (SpO2) 401 may be directly measured by the PO sensor 113, and a heart rate variability 405 may be directly measured by the ECG sensor 114. A snoring 403 may be directly measured by a audio recording 406 by the audio sensor 115.

However, in a state in which the PO sensor 113, the ECG sensor 114, and the audio sensor 115 are omitted or not used in the electronic device 10, it may be difficult to directly measure the blood oxygen saturation (SpO2) 401 and the snoring 403. In addition, when the acceleration sensor 111, the PPG sensor 112, the PO sensor 113, the ECG sensor 114, and the audio sensor 115 are all operated, the battery life may be reduced because the power consumption of the electronic device 10 is large, and memory resources may be wasted.

It is known that there are direct and indirect correlations between various physiological parameters in humans. For example, the correlation exists among heart rate and respiratory cycle, heart rate variability (HRV) and systolic and diastolic blood pressure values and trends, respiratory rate and heart rate variability (HRV) and various types of respiratory disorders (e.g., sleep apnea-hypopnea syndrome, snoring, and acute respiratory distress syndrome). The physiological parameters connected by dotted lines in FIG. 4 includes an interdependent correlation.

Accordingly, although the PO sensor 113, the ECG sensor 114, and the audio sensor 115 are omitted or not used, the electronic device 10 may estimate the physiological parameters such as the blood oxygen saturation (SpO2) 401, a sleep apnea/hypopnea syndrome 402, the snoring 403, and a heart rate disease 404 correlated with the bio-signals of the acceleration sensor 111 and the PPG sensor 112.

In other words, even when the electronic device 10 does not include the PO sensor 113 configured to directly measure the blood oxygen saturation, the processor 160 may estimate the blood oxygen saturation from data of other biosensors included in the electronic device 10.

In addition, the electronic device 10 may analyze the estimated physiological parameters. If necessary, the electronic device 10 may activate the PO sensor 113, the ECG sensor 114 and/or the audio sensor 115, or inform a user that the connection with the PO sensor 113, the ECG sensor 114 and/or the audio sensor 115 is required.

As mentioned above, by estimating various physiological parameters from some directly measured bio-signals, the electronic device 10 may provide various physiological information to the user even without many biosensors. In addition, the method for the electronic device 10 may analyze the estimated physiological parameter and activate the additional sensor only when necessary. Accordingly, the battery life may be increased, and the memory resources may be saved.

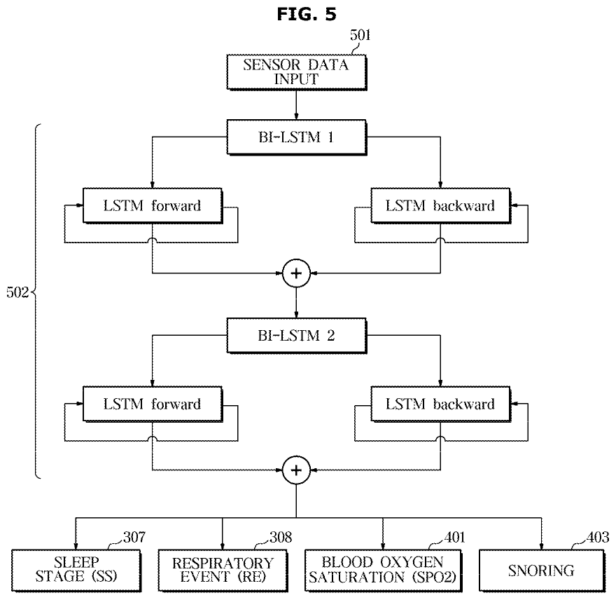

FIG. 5 is a diagram illustrating an example of a neural network architecture used to estimate the physiological parameters according to various embodiments.

Referring to FIG. 5, the processor 160 of the electronic device 10 may estimate various physiological parameters from data of the sensor 110 using the artificial intelligence model. FIG. 5 illustrates a long short-term memory (LSTM) model 502 among artificial intelligence models available in the electronic device 10.

In response to sensor data being input to the artificial intelligence model 502 (501), the artificial intelligence model 502 may process the sensor data so as to estimate the physiological parameters such as the sleep stage 307, the respiratory event 308, the blood oxygen saturation (SpO2) 401, and the snoring 403.

The LSTM model 502 is a type of a recurrent neural network (RNN) model. The RNN is a model that processes inputs and outputs in sequence units. The sequence refers to related sequence data, and may be a neural network model suitable for time series data. The RNN is suitable for estimation of physiological parameters because of temporal sequence processing and the possibility of hidden dependency analysis. The RNN shows promising results in ECG and PPG data processing, sleep stage analysis, and heart failure detection.

Bi-LSTM is a bidirectional LSTM. The Bi-LSTM includes forward LSTM and backward LSTM. The LSTM model 502 illustrated in FIG. 5 may include two stages of Bi-LSTM.

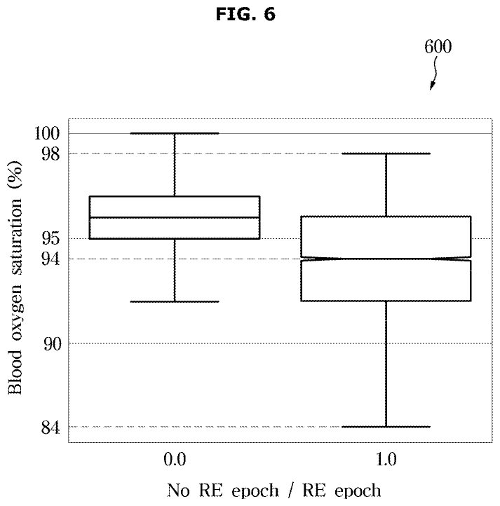

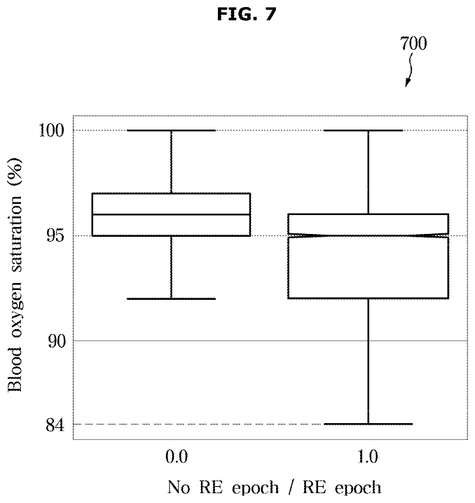

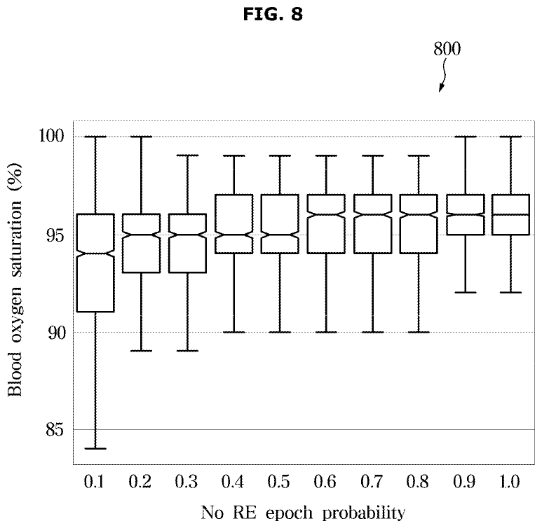

FIG. 6 is a graph illustrating a correlation between a respiratory event during sleep and a blood oxygen saturation according to various embodiments. FIGS. 7 and 8 are graphs (700, 800) illustrating a blood oxygen saturation estimated from a respiratory event during sleep by a method according to various embodiments.

Referring to FIGS. 6 and 7, No RE epoch is indicated as 0.0, indicating that a respiratory event such as apnea or hypopnea is not present. RE epoch is indicated as 1.0, indicating that a respiratory event such as apnea or hypopnea is present. A box and whisker plot shows the interquartile range (minimum, first quartile, median, third quartile, and maximum). A vertical line is the median.

In FIG. 6, a blood oxygen saturation in the presence of the respiratory event is lower than a blood oxygen saturation in the absence of the respiratory event that is a normal case. The median of actual values of the blood oxygen saturation in the presence of the respiratory event is 94%, and a range of the actual values is from 84% to 98%.

FIG. 7 illustrates that the estimated blood oxygen saturation in the presence of the respiratory event is similar to the actual value. The median of the estimated blood oxygen saturation is 95%, and the range of the estimated values is from 84% to 100%.

In addition, as shown in FIG. 8, a probability of the respiratory event may be estimated, and a blood oxygen saturation according to the probability of the respiratory event may also be estimated. A probability greater than 0.5 indicates the absence of the respiratory event, and a probability less than or equal to 0.5 indicates the presence of the respiratory event. As the probability decreases from 1 to 0, it is assumed that the blood oxygen saturation also decreases. That is, it can be seen that the estimation based on the correlation between the respiratory event and the blood oxygen saturation is accurate and efficient.

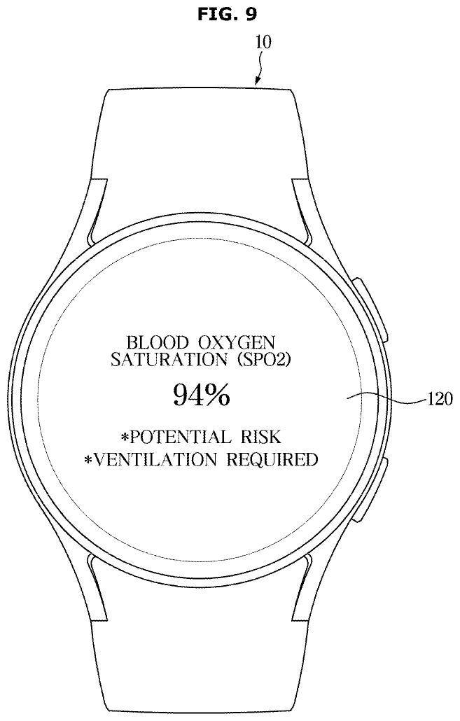

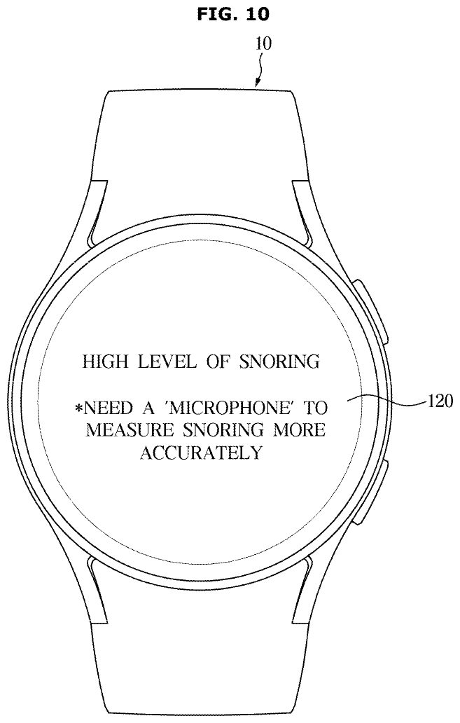

FIGS. 9 and 10 are diagrams illustrating an example of providing the estimated physiological parameter and information thereon by the electronic device according to various embodiments.

Referring to FIGS. 9 and 10, the display 120 of the electronic device 10 may output at least one piece of information about the bio-signal of the user, the first physiological parameter, and/or the second physiological parameter. The information about the second physiological parameter may include personalized feedback information based on the analysis of the second physiological parameter that changes over time. The personalized feedback information may include at least one piece of potential risk information about the physiological condition of the user or recommended activity information about the physiological condition of the user.

Further, the information about the second physiological parameter may include information about an additional sensor. The processor 160 may determine whether an additional sensor is needed for directly measuring the second physiological parameter, by comparing the estimated second physiological parameter with the predetermined reference value, and the processor 160 may control the display 120 to provide information about the additional sensor. The processor 160 may determine a point of time in which an operation of the additional sensor is required.

For example, as shown in FIG. 9, the display 120 of the electronic device 10 may display that the estimated blood oxygen saturation level is 94%, a warning message informing that a potential risk exists, and recommended activity information informing that ventilation is required. In addition, because the estimated blood oxygen saturation (SpO2) is lower than the reference value (e.g., 95%), information about the additional sensor indicating that the direct measurement by the PO sensor 113 is required may be provided on the display 120.

As another example, as shown in FIG. 10, based on the estimated snoring level being high, the display 120 of the electronic device 10 may display information about the additional sensor indicating that a microphone, which is the audio sensor, is needed to more accurately measure the snoring.

As is apparent from the above description, an electronic device and a control method thereof may estimate an interdependent physiological parameter including a correlation with a physiological parameter directly obtainable from a sensor.

Further, an electronic device and a control method thereof may provide various physiological information to a user even without many biosensors by estimating various physiological parameters from some directly measured bio-signals.

Further, an electronic device and a control method thereof may activate an additional sensor or provide information on the additional sensor if necessary, by analyzing estimated physiological parameters. Accordingly, battery life may be increased, and memory resources may be saved.

The various embodiments and the terms used therein are not intended to limit the technology disclosed herein to specific forms, and the disclosure should be understood to include various modifications, equivalents, and/or alternatives to the corresponding embodiments. In describing the drawings, similar reference numerals may be used to designate similar elements. A singular expression may include a plural expression unless they are definitely different in a context. The expressions “A or B,” “at least one of A or/and B,” or “one or more of A or/and B,” and the like used herein may include any and all combinations of one or more of the associated listed items. Herein, the expressions “a first”, “a second”, “the first”, “the second”, etc., may simply be used to distinguish an element from other elements, but is not limited to another aspect (importance or order) of elements. When an element (e.g., a first element) is referred to as being “(functionally or communicatively) coupled,” or “connected” to another element (e.g., a second element), the first element may be connected to the second element, directly (e.g., wired), wirelessly, or through a third component.

As used herein, the term “module” may refer to a unit that includes one or a combination of two or more of hardware, software, or firmware or any combination thereof. A “module” may be interchangeably used with terms such as, for example, unit, logic, logical block, component, or circuit. The module may be a minimum unit or part of an integrally constructed part. The module may be a minimum unit or part of performing one or more functions. The “module” can be implemented mechanically or electronically. For example, a “module” may be implemented in the form of an application-specific integrated circuit (ASIC).

Various embodiments of the present disclosure may be implemented as software including one or more instructions stored in a storage medium (e.g., a memory) readable by a machine (e.g., an electronic device). For example, a processor of an electronic device may call at least one instruction among one or more instructions stored in a storage medium and execute the instruction. This makes it possible for the device to be operated to perform at least one function according to the called at least one instruction. The one or more instructions may include code generated by a compiler or code executable by an interpreter. Storage medium readable by machine, may be provided in the form of a non-transitory storage medium. The “non-transitory” storage medium is a tangible device and may not contain a signal (e.g., electromagnetic wave), and this term includes a case in which data is semi-permanently stored in a storage medium and a case in which data is temporarily stored in a storage medium.

The method according to the various disclosed embodiments may be provided by being included in a computer program product. Computer program products may be traded between sellers and buyers as commodities. Computer program products are distributed in the form of a device-readable storage medium (e.g., compact disc read only memory (CD-ROM)), or are distributed directly or online (e.g., downloaded or uploaded) between two user devices (e.g., smartphones) through an application store (e.g., Play Storer“′). In the case of online distribution, at least a portion of the computer program product (e.g., downloadable app) may be temporarily stored or created temporarily in a device-readable storage medium such as the manufacturer's server, the application store's server, or the relay server's memory.

According to various embodiments, each component (e.g., a module or a program) of the above-described components may include a singular or a plurality of entities, and some of the plurality of entities may be separately arranged in other components. According to various embodiments, one or more components or operations among the above-described corresponding components may be omitted, or one or more other components or operations may be added. Alternatively or additionally, a plurality of components (e.g., a module or a program) may be integrated into one component. In this case, the integrated component may perform one or more functions of each component of the plurality of components identically or similarly to those performed by the corresponding component among the plurality of components prior to the integration. Operations performed by a module, a program module, or other elements according to various embodiments of the disclosure may be executed sequentially, in parallel, repeatedly, or in a heuristic method. Also, a portion of operations may be executed in different sequences, omitted, or other operations may be added

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. A method of controlling an electronic device comprising:
obtaining a bio-signal from at least one sensor;
determining a first physiological parameter based on the bio-signal;
estimating a second physiological parameter comprising a specified correlation with the first physiological parameter; and
providing information about the estimated second physiological parameter.

2. The method of claim 1, wherein
the second physiological parameter comprises at least one of biometric data or a physiological condition correlated with the first physiological parameter and not obtained by the sensor.

3. The method of claim 1, wherein
the estimation of the second physiological parameter comprises estimating the second physiological parameter dependent on the first physiological parameter using an artificial intelligence model.

4. The method of claim 3, wherein
the estimation of the second physiological parameter comprises estimating a plurality of different second physiological parameters from the first physiological parameter using the artificial intelligence model.

5. The method of claim 3, wherein
the artificial intelligence model comprises a least one of a deep neural network (DNN) model, a convolutional neural network (CNN) model, a recurrent neural network (RNN) model, or a long short-term memory (LSTM) model.

6. The method of claim 1, wherein
the providing of the information about the second physiological parameter comprises:
determining whether an additional sensor is needed for directly measuring the second physiological parameter, by comparing the estimated second physiological parameter with a specified reference value; and
providing information about the additional sensor.

7. The method of claim 1, wherein
the obtaining of the bio-signal and the estimation of the second physiological parameter is performed at a specified interval.

8. The method of claim 7, wherein
the information about the second physiological parameter comprises personalized feedback information based on an analysis of the second physiological parameter that changes over time.

9. The method of claim 8, wherein
the personalized feedback information comprises at least one of potential risk information about a physiological condition or recommended activity information about the physiological condition.

10. The method of claim 1, further comprising:
pre-processing the bio-signal,
wherein the pre-processing comprises data filtering, noise removal, motion artifact removal, and normalization and standardization of personalized data for variability reduction.

11. An electronic device comprising:
a display:
at least one sensor configured to obtain a bio-signal; and
a processor electrically connected to the display and the at least one sensor, wherein the processor is configured to:
determine a first physiological parameter based on the bio-signal;
estimate a second physiological parameter comprising a specified correlation with the first physiological parameter; and
control the display to provide information about the estimated second physiological parameter.

12. The electronic device of claim 11, wherein
the second physiological parameter comprises at least one of biometric data or a physiological condition correlated with the first physiological parameter and not obtained by the sensor.

13. The electronic device of claim 11, wherein
the processor is configured to estimate the second physiological parameter dependent on the first physiological parameter using an artificial intelligence model.

14. The electronic device of claim 13, wherein
the processor is configured to estimate a plurality of different second physiological parameters from the first physiological parameter using the artificial intelligence model.

15. The electronic device of claim 13, wherein
the artificial intelligence model comprises a least one of a deep neural network (DNN) model, a convolutional neural network (CNN) model, a recurrent neural network (RNN) model, or a long short-term memory (LSTM) model.
